(19) United States
(12) Patent Application Publication
(10) Pub. No.: US 2007/0238996 A1
(43) Pub. Date: Oct. 11, 2007

(54) Portable ultrasonic device and method for diagnosis of dental caries

(75) Inventors: Wei Lin, Port Jefferson, NY (US); Mark S. Wolff, Setauket, NY (US); Anilkumar R. Dhundale, Stony Brook, NY (US)

Correspondence Address: THE FARRELL LAW FIRM, P.C., 333 EARLE OVINGTON BOULEVARD, SUITE 701, UNIONDALE, NY, 11553, US

(73) Assignee: The Research Foundation of State University of New York, Albany, NY (US)

(21) Appl. No.: 11/374,737

(22) Filed: Mar. 14, 2006

(51) Int. Cl. A61B 8/00

(52) U.S. Cl. 600/437

(57) ABSTRACT

Provided is an apparatus and method for the detection of dental caries. The apparatus generates longitudinal ultrasound waves that may be applied to any accessible surface of a tooth. The reflected ultrasound pulse echoes are collected and correlated with the incident pulse. The ultrasound pulse echoes from the front and rear surface of a dental cavity may be distinguished from other echoes, and provided in visual display to inform as to the size and location of the cavity.

### BACKGROUND

1. Field of the Invention

The present invention relates to an apparatus and method for the ultrasonic detection of dental caries using longitudinal ultrasonic waves.

2. Description of Related Art

Currently, X-ray technology is the routine tool to examine for dental caries. Dental radiographs, to find dental caries (decay), show density differences in tooth structure caused by loss of calcium. Dental radiographs, however, can only be utilized to look for caries on the two accessible side surfaces of teeth. The remaining structures, particularly the occlussal (biting) surface, frequently develop considerably large carious lesions that remain undetectable to physical examination with the dental probe or by radiographic examination. These devices may not detect a cavity until it is advanced, and therefore is not a good tool for early detection and early treatment. A dental radiograph is radioactive, so it carries some degree of risk. Dental radiography is also expensive and non-portable.

Dental cavities most commonly develop on a tooth's biting surface. However, most caries have small entry openings and a much larger void can exist under the biting surface. These cavities cannot be easily found with the commonly used dental probes or X-rays. An existing device, DIAGNOdent, KaVo and KaVo America Corp. (www.kavo.com, www.kavousa.com) utilizes a colorimetric approach for detecting cavities. While this improves early detection, it is may be limited in performance for early detection, for example, because of non-uniformities in tooth color that create false signals or mask real signals.

Ultrasonic detection of dental caries has been attempted as a method to overcome the above-mentioned deficiencies associated with conventional techniques for detecting caries. Bab et. al. (U.S. Pat. No. 6,162,177) presented a device and method for the ultrasonic detection of smooth surface lesions on tooth crown surfaces such as caries and tooth crown surface cracks on a tooth crown surface. Bab et al. provides an ultrasonic surface wave transmitter/receiver, capable of transmitting an ultrasonic surface wave along a tooth crown surface. Surface lesions exposed to ultrasonic surface waves create ultrasonic surface wave reflections, which may be received at the transmitter/receiver, thereby detecting the presence of a cavity. Ultrasonic surface waves are capable of only limited penetration into the tooth and therefore, are unable to detect deeply penetrating caries or to determine the size of the caries.

### SUMMARY OF INVENTION

To overcome at least the foregoing problems, the present invention provides an apparatus and method for performing measurements and displaying the size of dental caries. The invention provides for the use of longitudinal ultrasound waves, which have a tooth penetrating capacity, not provided by ultrasonic surface waves used by others.

The present invention provides an apparatus for the detection of dental caries that includes an ultrasound transmitter for generating and transmitting electric pulse; a transducer for generating ultrasound pulse from the electric pulse, transferring the ultrasound pulse to a tooth, receiving ultrasound pulse echoes from surfaces and internal structure interfaces in the tooth and converting the ultrasound pulse echoes into received electric pulses; an ultrasound receiver for collecting and amplifying the received electric pulses; a switch for transmitting the generated electric pulses from the ultrasound transmitter to the transducer, and for transmitting the received electric pulses from the transducer to the ultrasound receiver; an echo detection unit for receiving the electric pulse transmitted from ultrasound transmitter and the received electric pulses from the ultrasound receiver and correlating the transmitted pulse and the received electric pulses; a display for displaying the location and size of the dental cavity; and a controller for controlling the ultrasound transmitter, the ultrasound receiver, the switch, the echo detection unit and the display.

The present invention provides a method for detection of dental caries that includes generating an electric pulse; transmitting the electric pulse through a switch to a transducer; generating an ultrasound pulse in the transducer from the transmitted electric pulse; applying the ultrasound pulse to an exterior tooth surface; converting ultrasound pulse echoes from surfaces and internal structure interfaces in the tooth to electric pulses in the transducer; switching the switch to receive electric pulses from the transducer; collecting and amplifying the received electric pulses in the ultrasound receiver; detecting the received electric pulses and correlating the ultrasound pulse echoes with the transmitted ultrasound pulse; and determining the location and size of the cavity.

Various display methods may be provided to give the user, effective information about the size and location of the cavity.

### BRIEF DESCRIPTION OF THE DRAWINGS

The above and other aspects and advantages of the present invention will be more clearly understood from the following detailed description taken in conjunction with the accompanying drawings in which:

FIG. 1 is a block diagram of an ultrasonic apparatus for the detection of dental caries according to a first embodiment of the present invention; and

FIG. 2 illustrates the display of an ultrasound pulse (I) transmitted into a tooth structure and a first reflection (a) from the front of a dental cavity_ and a second reflection (b) from the back of a dental cavity.

### DETAILED DESCRIPTION OF PREFERRED EMBODIMENTS

Hereinafter, preferred embodiments of the present invention will be described with reference to the accompanying drawings. It should be noted that the similar components may be designated by similar reference numerals although they are illustrated in different drawings. Also, in the following description, a detailed description of known functions and configurations incorporated herein will be omitted when it may obscure the subject matter of the present invention.

A novel approach for detection of dental caries is the use of a modulated ultrasonic wave that is transmitted through the tooth. When an ultrasound wave enters the tooth under examination, wave reflections occur at any density change interface such as the enamel-dentin interface, as well as at the dentin-pulp interface. These reflections, that are displaced in relation to time, make it possible to measure the thickness of the enamel and dentin, which are important indexes of tooth quality. In addition, when a dental cavity occurs inside a tooth, the cavity, because it is fluid filled and therefore of different density in comparison with surrounding tissue, will cause a change in the pattern of ultrasound reflection waves that will be detectable and quantifiable. This novel technology is capable of using an ultrasound wave to identify and estimate the size of a dental cavity as small as 1/10 of a millimeter in diameter.

Longitudinal ultrasound pulses, at a preferred frequency of 10 MHz are applied by the transducer at any accessible tooth surface. Echoes of the incident ultrasound pulse are reflected from internal tooth density interfaces including the front and back of the cavity. The ultrasound pulses are converted to echo electric pulses by the transducer, switched so as to be collected and amplified by the ultrasound receiver and the echoes from the front and rear surfaces of the dental caries are correlated by the echo detection unit and controller to distinguish them from the other echoes from other surfaces and interfaces of the tooth.

One potential issue is weakened ultrasound wave conduction because of air present in the cavity. Air is not as good a conductor of waves and a weak signal would penetrate the air, travel across the cavity and reflect off the opposite solid dentin or enamel surface. When waves enter the cavity if fluid is present much better conductance will occur and the wave reflected from the front and back end of the cavity will be detected as a stronger signal. A simple mouth wash with water prior to scanning should fill all cavities with fluid.

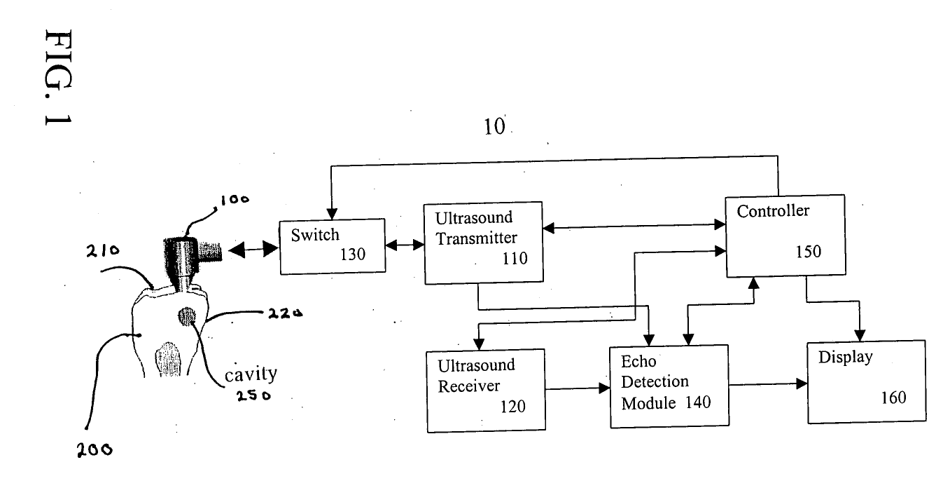

An apparatus according to the first embodiment of the invention is shown in FIG. 1. A dental caries detection apparatus 10 consists of a transducer 100, an ultrasound transmitter 110, an ultrasound receiver 120, a switch 130, an echo detection module 140, a controller 150, and a display 160. The dental caries detection apparatus 10 is applied to a tooth 200 to sense a dental cavity 250.

The ultrasound transmitter 110 generates an electric pulse in the form of a short pulse or a modulated signal to drive the transducer 100 to apply the ultrasound pulse to the tooth 200. The controller 150 determines a rate and energy of the pulse or signal. In a modulation mode, the controller also defines the signal format, e.g. frequency sweep signal with known duration and start/end frequencies. Ultrasound waves with a frequency of 1 MHz to 20 MHz may be used, but 10 MHz is the preferred frequency due to superior signal correlating capability in this range.

The transducer 100 converts the electric pulse or modulated ultrasound signal to an ultrasound pulse to either the occlussal (biting) surface 210 or a side surface 220 of the tooth 200.

The transducer 100 can be a two dimensional transducer array with N×N elements, with a preferred value of N=3. The maximum value of N is determined by the minimum achievable size of the transducer element. Each element serves as an individual ultrasound transducer, which has the capability of transmitting and receiving ultrasound signals. The activation of the transducer 100 is controlled by the switch 130 and the controller 150. By activating each element, the location of the ultrasound signal reflection and hence the cavity surface can be identified.

Alternatively, the transducer 100 can be a micro mechanical device with one transducer element mounting on a two dimensional (2-D) micro-stage. The movement of the stage is driven by two step motors or their equivalent micro positioning device. The control signal to the moving stage is sent from the controller 150 via the switch 130 to the transducer 100. The location of the transducer 100 is determined by the controlled positioning of the 2-D stage.

The switch 130, under the control of the controller 150, serves as a router, directing the electric pulse from the ultrasound transmitter 110 to the transducer 100 where it is transformed into an ultrasound pulse. The switching function is controlled by the controller 150. The ultrasound waves pass from the transducer 100 through one of the occlussal surface 210 and the side surface 220 into the tooth 200 where some of the energy of the incident wave is reflected, as ultrasound echoes, by the internal structures of the tooth 200. The signal reflection will be discussed later in more detail. The transducer 100 converts the ultrasound echoes back into electric pulses. The switch 130, under the control of the controller 150, directs the echo electric pulses to the ultrasound receiver 120.

In a transducer array mode, the switch 130 multiplexes the ultrasound electric pulses to the individual transducer element. The controller 150 provides address signals to the switch 130 to determine which element is activated. In the micro-stage mode, the controller 150 sends position pulses to the micro-positioning device via the switch 130 to move the transducer 100 to a known position.

The ultrasound receiver 120 collects and amplifies the received electric pulses from the transducer for analysis. In the preferred embodiment, the gain of the receiver is programmable in a range of 0 to 60 dB. In order to compensate for ultrasound energy attenuation in the tooth, the gain is controlled according to the time delay, i.e. the gain is increased for the signal coming from deep inside the tooth.

The echo detection module 140 receives the amplified received electric pulses and the electric pulses from the ultrasound transmitter 110 and identifies the location at which the echoes were generated from the different locations in the tooth. Cross correlation is one of the methods used to detect the significant echoes from the internal tooth structure. Echo signals are the reflections of the transmitted ultrasound signals delivered to the tooth. Therefore, the echo signals and the transmitted signals are highly correlated to each other. The signal that drives the ultrasound transmitter can be used as a template to correlate the echo signal. When the position of the template lines up with an echo signal, the correlation output will be a maximum. By identifying the maximums, which are the peaks of the cross correlation output, the echo positions can be detected. The ultrasound signal that drives the transmitter can be a predefined signal that yields the best correlation results. One such signal will be frequency sweep signal, where its correlation output will be a narrow pulse that can be easily identified. In this signal format, all the frequency components in the frequency sweep signal will be lined up at the same time point from the correlation process. This will generate a short pulse at that time point as compared to the long trail of frequency sweep signal whose frequency components are distributed evenly throughout the time duration of the signal. Phase locking is another technique to locate the ultrasound echoes from the dental cavity. When the ultrasound signal is in the format of tone burst, a series of sinusoidal cycles of a specific frequency, phase locking can detect the phase of the ultrasound echo signals with respect to the referenced signal, which bears the same frequency as the tone burst. The detected phase value can be converted to a time location of the received ultrasound signals.

When the ultrasound waves impinge on a surface of the tooth, some of the energy of the incident wave is reflected in the form of echoes. Prominent echoes are returned from the edges of the cavity, in addition to those from the tooth surface, the enamel-dentin interface, and the dentin-pulp interface. The echoes may be shifted in magnitude, direction and time due to the complex internal structure of the tooth and the cavity. The location and size of the cavity can be obtained according to the location of the echoes from the abnormal sites using the abovementioned techniques.

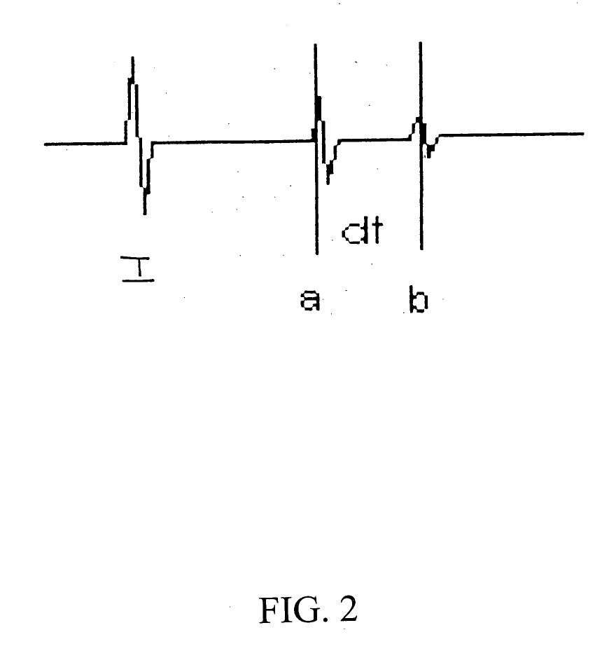

FIG. 2 illustrates an ultrasound pulse transmitted into a tooth structure, and a first and a second reflection from a dental cavity. Referring to FIG. 2, the first pulse I represents the incident ultrasound wave. The pulses a and b represent the echoes from the front and back of the cavity, and “dt” represents the time difference between the two echo pulses. The size of the cavity may be calculated based on the time difference between the two pulses using simple time calculations, such as s=v*dt/2 where s is the size of the cavity, v is a known speed of the ultrasound pulse in a cavity in the fluid filled medium of a cavity.

The controller 150 coordinates the generation and receipt of the ultrasound pulse, the performance and analysis of the collected data, and the generation of a report based on the evaluation.

If a transducer to be used is a micro mechanical device with one transducer element mounting on a two dimensional micro-stage, the control signals to the moving micro-stage is sent from the controller 150 via the switch 130 to the transducer 100. The controller 150 determines the rate and energy of the ultrasound signals generated in the ultrasound transmitter 110. In the modulation mode, the controller 150 also defines a signal format, e.g. frequency sweep signal with known duration and start/end frequencies.

The raw image of the ultrasound signal can be displayed on a display 160 to help the physician position the probe. An analysis result of the exam will also be displayed. Display methods can include performing the calculation of the dimension of the cavity by the controller 150 and the direct display of the cavity dimension on the screen. Further, the measuring the cavity can be done from several different positions on the external surface of the tooth such that a three-dimensional view of the cavity may be displayed. Still further, the display screen can display the cavity with respect to the major internal and external surfaces of the affected tooth.

While the invention has been shown and described with reference to a certain preferred embodiment thereof, it will be understood by those skilled in the art that various changes in form and details may be made therein without departing from the spirit and scope of the invention as defined in the appended claims.

We claim:

1. A method for detecting dental caries in a tooth, comprising the steps of:
generating an electric pulse;
transmitting the electric pulse through a switch to a transducer;
converting the electric pulse to an ultrasound pulse in the transducer;
applying the ultrasound pulse to an exterior tooth surface;
converting ultrasound pulse echoes from surfaces and internal structure interfaces in the tooth to electric pulses in the transducer;
switching the switch to receive the electric pulses from the transducer;
collecting and amplifying the received electric pulses in the ultrasound receiver;
correlating the ultrasound pulse echoes with the applied ultrasound pulse; and
determining the location and size of a dental cavity based on the correlation results.

2. The method for detecting dental caries as claimed in claim 1, wherein the ultrasound pulse generated in the transducer is a longitudinal wave.

3. The method for detecting dental caries as claimed in claim 2, wherein the ultrasound pulses generated in the transducer are at a frequency in the 1 MHz to 20 MHz range.

4. The method for detecting dental caries as claimed in claim 3, wherein the ultrasound pulses generated in the transducer are at a preferred frequency of 10 MHz.

5. The method for detecting dental caries as claimed in claim 3, wherein the ultrasound pulses generated in the transducer are modulated pulses.

6. The method for detecting dental caries as claimed in claim 4, wherein the ultrasound pulses generated in the transducer are modulated pulses.

7. The method for detecting dental caries as claimed in claim 5, wherein the ultrasound pulses generated in the transducer are modulated sinusoidal pulses.

8. The method for detecting dental caries as claimed in claim 6, wherein the ultrasound pulses generated in the transducer are modulated sinusoidal pulses.

9. The method for detecting dental caries as claimed in claim 1, wherein applying the ultrasound pulse to the tooth by the transducer includes positioning the transducer on at least one of the occlussal and side surfaces of the tooth.

10. The method for detecting dental caries as claimed in claim 1, wherein applying the ultrasound pulse to the exterior tooth surface includes:
activating individual elements of a two-dimensional transducer array with N×N elements using the switch controlled by the controller.

11. The method for detecting dental caries as claimed in claim 1, wherein applying the ultrasound pulse to an exterior surface of the tooth includes:
mounting a single element transducer on a micro mechanical device;
positioning the single element transducer at desired locations on the exterior surface of the tooth using the micro mechanical device; and
activating the transducer using the switch controlled by the controller.

12. The method for detecting dental caries as claimed in claim 1, wherein the gain of the receiver is programmed to compensate for ultrasound energy attenuation in the tooth according to a time delay experienced in receiving the ultrasound pulse echo.

13. The method for detecting dental caries as claimed in claim 1, wherein correlating the ultrasound pulse echoes with the transmitted ultrasound pulse includes using cross correlating techniques.

14. The method for detecting a dental caries as claimed in claim 13, wherein the cross correlating technique includes a phase loop locking technique performed by the echo detection unit.

15. The method for detecting dental caries as claimed in claim 12, wherein cross-correlating techniques are used to determine a first received ultrasound pulse echo representing a front surface of the cavity and a second received ultrasound pulse echo representing the back surface of the cavity.

16. The method for detecting dental caries as claimed in claim 1, wherein determining the location and size of the dental cavity includes:
receiving a first ultrasound pulse echo representing a front surface of the dental cavity and second ultrasound pulse echo representing the back surface of the dental cavity;
measuring the time (dt) between the first and second ultrasound pulse echoes; and
determining the size of the dental cavity based on
s=v*dt/2
where s is the size of the cavity, v is a known speed of the ultrasound pulse in a fluid medium filled in a cavity, and dt is the measured time between the first and second ultrasound pulses.

17. The method for detecting dental caries as claimed in claim 1, further comprising the step of displaying information related to at least one of the size and location of the cavity.

18. The method for detecting dental caries as claimed in claim 17, wherein displaying information includes providing a visual display of the amplitude of the applied ultrasound pulse, the first received ultrasound pulse echo and the second received ultrasound pulse echo in a plot versus time.

19. The method for detecting dental caries as claimed in claim 17, wherein displaying information includes providing a direct display of the cavity dimensions on the screen.

20. The method for detecting dental caries as claimed in claim 17, wherein displaying information includes displaying a three-dimensional image of the cavity.

21. The method for detecting dental caries as claimed in claim 17, wherein displaying information includes displaying an image of the cavity with respect to an image of major structures of the affected tooth.

22. An apparatus for the detection of dental caries comprising:
an ultrasound transmitter for generating and transmitting electric pulses;
a transducer for generating ultrasound pulse from the transmitted electric pulse, transferring the ultrasound pulse to the tooth, receiving ultrasound pulse echoes from the tooth, and converting the ultrasound pulse echoes into received electric pulses;
an ultrasound receiver for receiving and amplifying the received electric pulses;
a switch for directing the electric pulses to the transducer from the ultrasound transmitter and for directing the received electric pulses from the tooth to the ultrasound receiver;
an echo detection unit for receiving the electric pulses transmitted from the ultrasound transmitter and the amplified electric pulses from the ultrasound receiver, and correlating the ultrasound pulse echoes from the tooth with the generated ultrasound pulses;
a data display for displaying cavity information; and
a controller for controlling operation of the ultrasound transmitter, the ultrasound receiver, the switch, the echo detection unit and a data display.

23. The apparatus as in claim 22, wherein the ultrasound pulses converted by the transducer are longitudinal waves.

24. The apparatus as claimed in claim 23, wherein the transducer generates ultrasound pulses in the 1 MHz to 20 MHz frequency range.

25. The apparatus as claimed in claim 24, wherein the transducer generates modulated ultrasound pulses.

26. The apparatus as claimed in claim 25, wherein the transducer generates modulated sinusoidal ultrasound pulses

27. The apparatus as claimed in claim 23, wherein the transducer generates ultrasound pulses preferentially at 10 MHz.

28. The apparatus as claimed in claim 27, wherein the transducer generates modulated ultrasound pulses.

29. The apparatus as claimed in claim 28, wherein the transducer generates modulated sinusoidal ultrasound pulses.

30. The apparatus as claimed in claim 22, wherein the ultrasound pulse generated is a customized pulse for which the frequency and pulse shape is modifiable to optimize echo correlation.

31. The apparatus for detecting dental caries as claimed in claim 22, wherein the transducer applies the ultrasound pulse to at least one of the occlussal and side surfaces of the tooth.

32. The apparatus for detecting dental caries as claimed in claim 22, wherein the transducer for applying the ultrasound pulse to the exterior surface of the tooth includes:
a two-dimensional transducer array with N×N elements; and
individual elements of the transducer array are activated using the switch controlled by the controller.

33. The apparatus for detecting a dental cavity as claimed in claim 22, wherein the transducer for applying the ultrasound pulse to the exterior surface of the tooth includes:
a single element transducer mounted on a micro mechanical device, wherein the controller controls the positioning of the single element transducer at desired locations on the exterior surface of the tooth and activates the elements of the transducer using the switch.

34. The apparatus for detecting dental caries as claimed in claim 22, wherein the gain of the receiver is programmable to compensate for ultrasound energy attenuation in the tooth according to a time delay experienced in receiving the ultrasound pulse echo.

35. The apparatus for detecting dental caries as claimed in claim 22, wherein the gain of the receiver is programmable to compensate for ultrasound energy attenuation in the tooth is programmable in the range of 0 db to 60 db.

36. The apparatus for detecting dental caries as claimed in claim 22, wherein the controller determines the rate and energy of the ultrasound pulses and defines a signal format.

37. The apparatus for detecting dental caries as claimed in claim 36, wherein the echo detection unit correlates the ultrasound pulse echoes from the tooth with the transmitted ultrasound pulses using cross correlation techniques.

38. The apparatus for detecting dental caries as claimed in claim 37 wherein the cross correlation techniques used by the echo detection unit include phase loop locking.

39. The apparatus for detecting a dental cary as claimed in claim 38, wherein the echo detection unit:
receives a first ultrasound pulse echo representing a front surface of the cavity and a second ultrasound pulse echo representing a back surface of the cavity;
measures the time (dt) between the first ultrasound pulse echo and the second ultrasound pulse echo; and
determines the size of the cavity using
s=v*dt/2
where s is the size of the cavity, v is a known speed of the ultrasound pulse in a fluid medium filled in a cavity, and dt is the measured time between the first and second ultrasound pulse echoes.

40. The apparatus for detecting dental caries as claimed in claim 22, wherein the echo detection unit correlates the ultrasound pulse echoes from the tooth with the transmitted ultrasound pulses using cross correlation techniques.

41. The apparatus for detecting dental caries as claimed in claim 40, wherein the cross correlation techniques used by the echo detection unit determine a first received ultrasound pulse echo representing a front surface of the cavity and a second received ultrasound pulse echo representing a back surface of the cavity.

42. The apparatus for detecting dental caries as claimed in claim 22, wherein the display provides display information relating to at least one of size and location of the cavity.
